(19) United States
(12) Patent Application Publication
(10) Pub. No.: US 2007/0118093 A1
(43) Pub. Date: May 24, 2007

(54) HIGH-SPEED JET DEVICES FOR DRUG DELIVERY

(76) Inventors: Marcio G. von Muhlen, Cambridge, CA (US); Laleh Jalilian, St. Louis, MO (US); Menzies Chen, San Diego, CA (US); Ravi Srinivasan, Mountain View, CA (US); Daniel A. Fletcher, Berkeley, CA (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 11/425,371

(22) Filed: Jun. 20, 2006

Related U.S. Application Data
(60) Provisional application No. 60/692,685, filed on Jun. 20, 2005.

(51) Int. Cl. A61M 31/00

(52) U.S. Cl. 604/500

(57) ABSTRACT

A method and apparatus for performing jet injections using piezoelectric technology. A jet injector apparatus includes a reservoir having a variable capacity for holding a liquid to be delivered to a biological tissue via a jet injection. A nozzle is disposed at a first end of the reservoir with at least one opening for allowing the liquid to be expelled from the reservoir. The capacity of the reservoir may be altered by the actuation of a piezoelectric transducer located at a second end of the reservoir

### CROSS-REFERENCE TO RELATED APPLICATIONS

This application claims the benefit of U.S. Provisional Application Ser. No. 60/692,685, filed Jun. 20, 2005, which is incorporated herein by reference in its entirety.

### BACKGROUND

1. The Field of the Invention

The present invention relates generally to medical devices including high-speed jet injection devices. More specifically, the present invention relates to methods and systems for transdermal delivery of drugs and other materials to biological tissue through use of pulsatile jets employing a piezoelectric actuator.

2. The Relevant Technology

One common method of delivering medication into the human body is via pills that are taken orally. The drugs in the pills are absorbed by the gastro-intestinal (GI) tract into the blood stream for systemic delivery. Unfortunately, a large fraction of the drug candidates either do not have the right solubility to be absorbed by the GI tract or are destroyed by the digestive secretions.

Transdermal delivery of drugs provides several advantages over oral pills. Although transdermal drug delivery has been in existence for two decades and provides a highly effective way of delivering drugs systemically, only a small number of drugs can be passively absorbed through the skin at therapeutic levels. Transdermal patches have many benefits, including avoiding first pass metabolism, ability to maintain smooth dosage levels and avoid the peaks and troughs experienced with pills, injections, and pulmonary and transmucosal drug delivery methods. They are a convenient dosage vehicle and achieve high levels of patient compliance. Despite the advantages that patches have, there are approximately only ten drugs that are commercially available in patch formats.

Evolved to impede the flow of toxins into the body, the skin has very low permeability to foreign molecules. The main barrier to diffusion of pharmaceuticals is the outermost layer of skin, the stratum corneum. The stratum corneum consists of densely packed keratinocytes (flat dead cells filled with keratin fibers) surrounded by lipid bilayers, which are highly ordered. This creates an effective barrier to drug transport. A few small molecules have been successfully transported across the skin by passive diffusion in therapeutic quantities. However, macromolecules are typically 10 to 100 times heavier than the small molecule transdermal successes. The large mass as well as the limited solubilities of the macromolecules in the lipid bilayers, limits their transdermal diffusion rates. As a result, most macromolecules have to be injected.

Various methods for enhancing transdermal drug flux have been attempted including using chemical enhancers. Their development has been hampered by skin irritation and incompatibility with the drug formulation. Other approaches have been attempted to disrupt the stratum corneum barrier. Microneedles that penetrate the stratum corneum have been proposed for painless macromolecule transdermal drug delivery. Low-frequency ultrasound has been shown to enhance transdermal drug delivery by disrupting the lipid bilayers due to cavitation effects. After application of ultrasound, a patch with the desired therapeutic is worn to deliver the drug through passive diffusion. Local heating to burn off the stratum corneum (thermoporation) has been proposed for transdermal drug delivery. These techniques often suffer from the absence of time-dependent dosage delivery, which is important to several therapeutics including insulin.

Needleless injectors are one form of hypodermic syringe replacement. They use high-speed jets that penetrate the stratum corneum and deliver a bolus of drug in a very short period of injection time (typically less than 500 ms). The jet injectors have, however, failed to gain a significant market share mainly because the injection can be very painful for some patients and also because of the large variability in dosage.

The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

One embodiment is directed to a jet injector apparatus. The apparatus includes a reservoir having a variable capacity for holding a liquid to be delivered to a biological tissue via a jet injection. A nozzle is disposed at a first end of the reservoir with at least one opening for allowing the liquid to be expelled from the reservoir. The capacity of the reservoir may be altered by the actuation of a piezoelectric transducer located at a second end of the reservoir.

Another embodiment of the invention is directed to a jet injector apparatus having a microliter syringe barrel. The microliter syringe barrel includes a reservoir for holding a liquid, such as a drug. A nozzle is disposed at a first end of the syringe barrel with at least one opening for allowing the liquid to be expelled from the reservoir of the syringe. The second end of the syringe barrel receives a plunger, which is operatively connected to a piezoelectric transducer. The plunger may be actuated by the piezoelectric transducer, thereby causing the liquid to be expelled from the reservoir in the form of a jet injection. The apparatus may further include an electronic circuit for controlling various properties of the jet injection. The electronic circuit may be used for actuating the transducer, and may include a power source, a solid state switch and a resistor.

Additional features will be set forth in the description which follows, and in part will be obvious from the description, or may be learned by the practice of the teachings herein. Features of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

To further clarify the features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates one embodiment of a piezoelectric jet injector apparatus;

FIG. 2 illustrates one embodiment of an electronic circuit used to control various properties of a piezoelectric jet injector apparatus; and

FIG. 3 illustrates an exemplary flow chart of a method for performing a jet injection.

### DETAILED DESCRIPTION

In the following detailed description of the preferred embodiments, reference is made to the accompanying drawings which form a part hereof, and in which are shown by way of illustration specific embodiments in which the invention may be practiced. It is to be understood that other embodiments may be utilized and structural changes may be made without departing from the scope of the present invention.

Embodiments of the present invention relate to a jet injector for providing transdermal inoculation of drugs, precision cutting of biological tissues, among other applications. The electronics used to drive the piezoelectric actuator improves ejection reliability through electronic control of jet speed and volume. The jet injector includes a reservoir having a variable capacity for holding a liquid to be delivered to a biological tissue via a jet injection. A nozzle is disposed at a first end of the reservoir with at least one opening for allowing the liquid to be expelled from the reservoir. A piezoelectric transducer is provided at a second end of the reservoir. When the piezoelectric transducer is actuated, the capacity of the reservoir is altered, thereby expelling the liquid in the form of a jet injection.

The jet injector ejects a small diameter, high velocity jet pulse of liquid that can be used as a carrier to deliver drugs or as a cutting tool. Embodiments of the present invention may serve as a replacement and/or a complement to hypodermic needles and other existing transdermal drug delivery devices, especially for delivery into soft and sensitive tissues where precise delivery is necessary. The nature of the jet also allows for its application as a precision cutting tool for biological tissue.

As used herein, the term “jet injection” refers to a small diameter, high velocity jet pulse of liquid used to penetrate a biological tissue without the use of a needle. A jet injection is typically produced by means of a “jet injector” device that uses high pressure to force the liquid from a small diameter nozzle. The biological tissue may include skin, an organ membrane, or any other physical diffusion barrier. Examples of jet injection include high-speed jet bolus delivery. The term “jet injection” may have a variety of applications, including a precision cutting tool for biological tissue, needle replacement for use in tattooing, transdermal delivery of drugs or other liquids, as well as other applications.

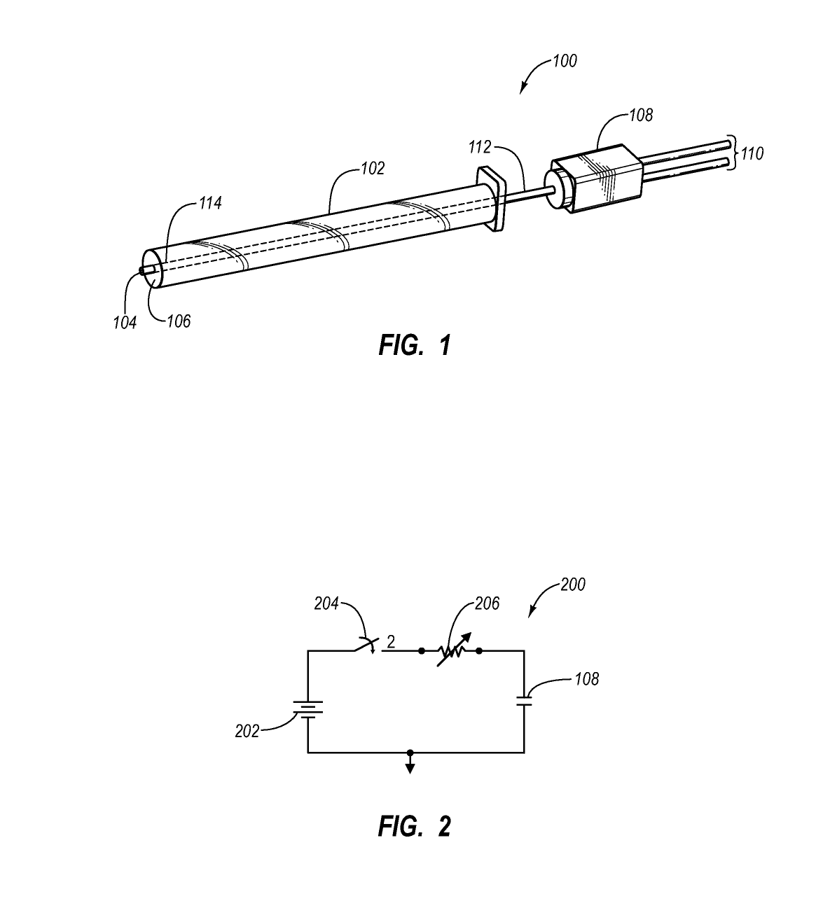

Referring now to FIG. 1, a more detailed example is illustrated using a diagrammed reference of a piezoelectric micro fluidic jet injector 100. A piezoelectric transducer 108 is held behind a modified micro liter capacity syringe barrel 102. The syringe barrel 102 contains a reservoir 114 for holding a liquid to be injected into a soft biological tissue of a human or animal. A nozzle 104 is coupled to one end 106 of the syringe barrel 102. For example, in one embodiment, the nozzle 104 is constructed from a heat-pulled glass pipette fashioned into a small diameter (e.g., 60-100 μm). In one embodiment, the nozzle 104 is adhered to the syringe tip 106, for example, using epoxy or similar adhesive. In one embodiment, the reservoir 114 may be continuously filled with a liquid by an external, pressurized fluid source through a separate port (not shown). Similarly, multiple drugs may be added through multiple ports to create a solution that may be injected.

The transducer 108 may be connected by wires 110 to a driver box, which is connected to a high voltage power supply. In one embodiment, the supply can be set between 0 to 150 volts to control expansion or displacement of the piezoelectric transducer 108.

The piezoelectric transducer 108 is coupled to a plunger 112, which travels through the syringe barrel 102 for controlling the volume of the internal reservoir 114. As the piezoelectric transducer 108 expands, the plunger 112 is rapidly pushed into the syringe barrel 102, thereby causing the fluid contained within the reservoir 114 to be expelled from the nozzle 104.

In one embodiment, the nozzle 104 is configured to have a diameter less than 80 μm. By providing a small diameter nozzle 104, the liquid jet injection minimizes the pain of drug delivery and offers precise cutting capabilities. By way of comparison, even the smallest conventional hypodermic needle diameters are typically larger than 200 μm. Since the cross-sectional area or of the point of entry of the jet injections is approximately equal to the square of the nozzle diameter, reducing the diameter of the nozzle by a factor of 4 results in 93.75% smaller cross-sectional area of the injection.

In FIG. 2, a simplified Resistance-Capacitance (RC) circuit 200 representing the driver box described above is shown. The RC circuit 200 may include, for example, a voltage source 202 that is placed in series with a switch 204, a resistor 206 and a piezoelectric transducer, represented by capacitor 108. Electrical energy from the voltage supply 202 is discharged to the piezoelectric transducer 108 when the device is triggered, causing the transducer 108 to expand. The circuit 200 illustrated in FIG. 2 is one example of a means for applying a high level of energy to the piezoelectric transducer 108 in a short period of time. The voltage source 202 stores a large amount of energy such that when the switch 204 is closed, the energy from the voltage source 202 is applied to the piezoelectric transducer 108, thereby causing the transducer to expand. In one embodiment, the switch 204 is a solid state switch for providing very rapid closing times. Referring again to FIG. 1, as the piezoelectric transducer 108 expands, the plunger 112 is pushed into the syringe barrel 102, thereby causing a jet injection to be driven out of the nozzle 104 at high speeds.

It one embodiment, the resistor 202 may include a variable resistor, such as a potentiometer. Adjusting the resistance and voltage of the RC circuit 200 allows the user to independently control the speed of transducer 108 and the length of transducer extension, respectively. In particular, the voltage level controls the length of transducer extension, thereby controlling the volume of the jet injection. The level of the resistance provided by the resistor 206 controls the time interval at which the energy from the voltage source 202 is transferred to the piezoelectric transducer 108, thereby controlling the velocity of the jet injection. For instance, when diameter of the nozzle 104 is kept constant at 69 microns, it has been shown that varying resistance and voltage to the circuit 200 allows the velocity of the jet stream to be controlled between 33 to 140 meters per second and volume ejected between 55 to 140 nanoliters. By way of comparison, jet injectors employed in intact printers generally generate jet speeds of approximately 5 m/s.

Independent control of the velocity and volume of the jet injection, as is provided in the circuit of FIG. 2, affords a higher degree of control over conventional jet injectors lacking independent volume and velocity control. For example, the ability to precisely control the volume, velocity, and the repetitive nature of the jet injection provide accurate injection doses and injection depth control. Advantageously, the parameters of a jet injector may be customized for the unique tissues and structural characteristics of each individual patient. The ability to rapidly and easily tailor a jet injection to a specific application and/or patient makes the jet injections described herein accessible to a larger population of patients.

The piezoelectric jet injector 100 of the present invention has a variety of applications. For example, the jet injector 100 may be used to provide transdermal inoculation of a drug or other fluid to a biological tissue. Alternatively, the jet injector 100 may be employed to cut biological tissue such as skin for applications in microsurgery, for extraction of biological fluids for diagnostics, and the like. In another embodiment, the jet injector 100 may be used as a needle replacement for use in tattooing. The micro fluidic jet injector 100 has applications in both human and veterinary areas.

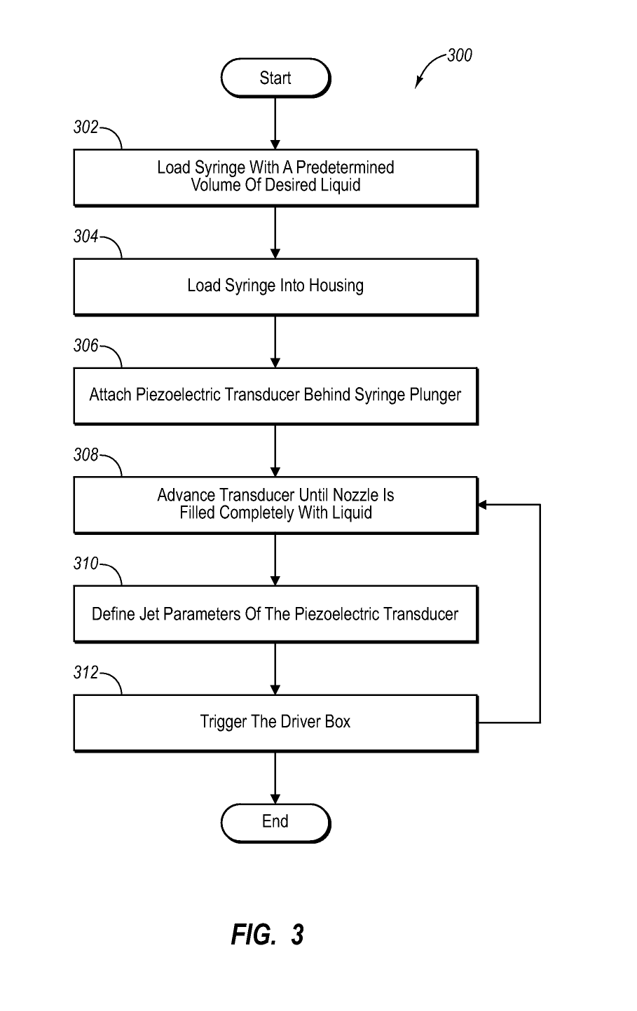

FIG. 3 illustrates one embodiment of a method 300 of operating a piezoelectric micro fluidic jet injector, such as the injectors illustrated in FIGS. 1-3. A reservoir of a syringe barrel is loaded 302 with a predetermined volume of desired liquid. When the syringe barrel is loaded, care should be taken so no air bubbles enter the liquid reservoir, as air bubbles reduce the pressure impulse generated by the movement of the plunger. Particular care should be taken when the liquid being loaded into the syringe barrel possesses certain fluid dynamic criteria, such as fluids having excessively high or low surface tension which may cause air bubbles to be pulled into the reservoir through the nozzle.

The syringe barrel is loaded 304 into a housing, and a piezoelectric transducer is attached 306 behind the plunger. The transducer is advanced 308 until the nozzle of the syringe is filled completely with the liquid previously loaded into the reservoir of the syringe. Prior to operating the piezoelectric micro fluidic jet injector, a user can define 310 parameters of the jet injector, such as the velocity of the jet injection, the volume of the jet injection, the frequency of the jet injection, and the like, by adjusting the voltage level of a voltage source, the circuit resistance of a driver box, and other controls.

After the target media is in place, the user may trigger 312 the driver box by applying the predetermined voltage to the piezoelectric transducer. Based on the parameters defined at 310, the driver box may raise the voltage applied to the piezoelectric transducer within 3 to 20 microseconds. The piezoelectric transducer converts the applied voltage into a mechanical extension. The degree of mechanical extension may vary from 5 to 20 microns depending on a number of factors, including voltage levels, size of the piezoelectric transducer, and the like. The force of the mechanical extension results in an acceleration of the plunger into the syringe barrel. Because the movement of the plunger alters the volume of the reservoir, the pressure inside the reservoir increases, for example, by approximately 10 MPa. The pressure differential results in a jet injection exiting the nozzle at high velocities. In one embodiment, the jet injection event lasts for approximately 1 ms, with decreasing jet velocities as the pressure inside the reservoir decreases.

Steps 308 through 312 may be repeated until the liquid contained within the reservoir falls below a predefined minimum level. If necessary, the positioning of the piezoelectric transducer in relation to the plunger and syringe barrel may be adjusted between each jet injection. The adjustment of the transducer position may be performed either manually or automatically. The device parameters used in this example are for a single embodiment of the invention and is included for illustration purposes only. Other device configurations working on the same principle but operating with different device parameters may also be built.

In one embodiment, technology for sensing skin or other biological tissue properties is integrated into the systems and methods described in FIGS. 1-3 for providing automatic adjustment of jet injection parameters to accommodate differing skin and biological tissue types.

In one embodiment, the jet injectors described in FIGS. 1-3 may further be equipped with a computer microprocessor for management of total dose, jet injection volume and velocity, as well as other jet injection parameters. A fully automated system may be completely self-sufficient, reducing or eliminating the need for human intervention for administering and/or monitoring drug deliveries.

In one embodiment, an array of transducers may be employed and incorporated into a medical adhesive patch to provide greater delivery volumes or to provide delivery over a wide area of tissue.

A piezoelectrically driven jet injector, such as those described in FIGS. 1-3, and the method described in FIG. 3 may be practiced in a number of ways. In one embodiment, a portable handheld jet injector may be constructed for use in a clinical or home environment to deliver concentrated solutions of drugs to sensitive tissues. In another embodiment, a jet injector may be integrated into a robotic surgical unit, wherein the injector may allow for targeted delivery to a specific location in a patient. As described previously, exemplary applications may include delivery of medication to a skin lesion or tumor, tattooing, use as a microsurgical cutting tool, and the like.

Most conventional commercial jet injectors are powered either by springs or compressed air. Consequently, many conventional jet injectors lack precise control of jet velocity, are limited to a small number of factory defined settings, and cannot be operated repetitively. Furthermore, many conventional commercial jet injectors lack the ability to independently control the velocity and volume of the jet injection. The present invention overcomes these shortcomings through the use of solid-state actuation and control, thereby providing for repetitive pulsatile jet injection, and independent control of the velocity and volume of the jet injection.

The exemplary embodiments described above may provide the ability to treat previously inaccessible tissues and diseases. The biological damage caused by needles limits their use to robust areas of the body. Tissues previously thought too sensitive for treatment by drug injection, such as the retinal arteries or the articular cartilage in small joints, could safely and reliably be treated using the techniques described herein. The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus, comprising:
a microliter syringe barrel having a reservoir therein for holding a liquid;
a nozzle disposed at a first end of the syringe barrel with at least one opening for allowing the liquid to be expelled from the reservoir;
a plunger received in a second end of the syringe barrel; and
a piezoelectric transducer operatively connected with said plunger for actuation of the plunger, the actuation of the plunger causing the liquid to be expelled from the reservoir in the form of a jet injection.

2. The apparatus as recited in claim 1, further comprising an electronic circuit for actuating the piezoelectric transducer, the electronic circuit comprising a power source in series with a solid state switch and a resistor, wherein an output of the electronic circuit is coupled to the piezoelectric transducer.

3. The apparatus as recited in claim 1, wherein a resistance value of the resistor is variable for controlling a velocity of the jet injection.

4. The apparatus as recited in claim 1, wherein a voltage level of the power source is variable for controlling a volume of the jet injection.

5. The apparatus as recited in claim 1, wherein a diameter of the nozzle is 100 micrometers or less.

6. The apparatus as recited in claim 1, wherein the jet injection is employed for providing transdermal inoculation of the liquid into a biological tissue.

7. The apparatus as recited in claim 1, further comprising at least one external pressurized fluid source coupled to the reservoir of the syringe barrel for providing a continuous supply of the liquid.

8. An apparatus, comprising a hollow structure for creating jet injections, the hollow structure having a nozzle on one end and a plunger inserted in the other end; wherein the plunger is actuated by a piezoelectric transducer and the piezoelectric transducer is driven by an electronics unit comprising a voltage source, a solid state switch, and a resistor.

9. The apparatus as recited in claim 8, wherein a volume of the jet injection is controlled independently from a velocity of the jet injection.

10. The apparatus as recited in claim 9, wherein the resistor has adjustable resistance, and wherein the level of resistance controls the velocity of the jet injection.

11. The apparatus as recited in claim 9, wherein the voltage source is variable, and wherein the level of the voltage source controls the volume of the jet injection.

12. A method for operating a piezoelectric jet injector, the method comprising:
loading a syringe barrel with a predetermined volume of a liquid, the syringe barrel having a nozzle on a first end for allowing the liquid to be expelled from the syringe barrel;
loading a plunger into a second end of the syringe barrel, wherein a piezoelectric transducer is positioned for actuating the plunger; and
applying a predetermined voltage to the piezoelectric transducer to actuate the plunger and expel the liquid from the nozzle in the form of a jet injection.

13. The method as recited in claim 12, further comprising advancing the plunger and the piezoelectric transducer until the nozzle of the syringe is filled completely with the liquid.

14. The method as recited in claim 12, further comprising defining jet injection parameters including a velocity of the jet injection and a volume of the jet injection.

15. The method as recited in claim 14, wherein the volume of the jet injection is controlled by adjusting the predetermined voltage, and wherein the velocity of the jet injection is controlled by adjusting a resistance of an electronic circuit employed for controlling the piezoelectric transducer.

16. The method as recited in claim 12, further comprising adjusting the positioning of the piezoelectric transducer in relation to the plunger between each successive application of the predetermined voltage to the piezoelectric transducer.
